(19) United States
(12) Patent Application Publication
(10) Pub. No.: US 2014/0017295 A1
(43) Pub. Date: Jan. 16, 2014

(54) HEMOSTATIC MATERIAL

(71) Applicant: JNC CORPORATION, Tokyo (JP)

(72) Inventors: AKIKO SHIMATANI, YOKOHAMA-SHI (JP); HIROAKI WAKAMOTO, TOKYO (JP); ERI IWAMOTO, YOKOHAMA-SHI (JP)

(21) Appl. No.: 13/938,238

(22) Filed: Jul. 10, 2013

(30) Foreign Application Priority Data
Jul. 11, 2012 (JP) 2012-155454

(51) Int. Cl. A61K 38/16; A61K 9/70; C07K 14/00

(52) U.S. Cl. CPC A61K 38/16; C07K 14/00; A61K 9/70
USPC 424/443; 530/350; 514/21.2

(57) ABSTRACT

A medical material having high hemostatic ability, which is easy to handle and shows improved productivity, is described.

A hemostatic material comprising a polypeptide having a peptide fragment represented by General Formula (1) below:

-(Pro-Y-Gly)ₙ- (1)

[wherein in General Formula (1), Y represents hydroxyproline or proline, and n represents an integer of 74 to 171].

### TECHNICAL FIELD

### Cross-Reference to Related Applications

This application claims the priority benefit of Japan application serial no. 2012-155454, filed on Jul. 11, 2012. The entirety of the above-mentioned patent application is hereby incorporated by reference herein and made a part of this specification.

The present invention relates to a hemostatic material comprising a collagen-like polypeptide.

### BACKGROUND ART

Collagen, which is a protein, is widely used as a general-purpose medical material. Naturally occurring type I collagen molecules have a characteristic primary structure composed of repeats of three amino acid residues, Gly-X-Y (wherein X and Y each represents various amino acids, and X is Pro and Y is Hyp in many cases). Three molecules of this polypeptide gather in the same direction to form a triple-helix tertiary structure, forming a collagen fiber.

On the other hand, polypeptide molecules composed of repeats of three amino acid residues, Pro-Y-Gly (wherein Y represents proline or hydroxyproline), which were created as collagen-like polypeptides (the so called synthetic collagens), are also reported to form a triple-helix structure (S. Sakakibara et al., Biochim. Biophys. Acta, 303, 198 (1973); T. Kishimoto et al., Biopolymers, 79, 163-172 (2005)).

In collagen-like polypeptides, unlike naturally occurring collagen, there is no risk of causing infectious diseases; the polypeptides can be stably supplied since they are industrially synthesized; their triple-helix structure is thermally stable; and the polypeptides are colorless and odorless. Because of these and other excellent characteristics, collagen-like polypeptides have been studied as various functional materials. For example, a property to cause platelet aggregation has been disclosed (WO 2008/075589).

Further, a hemostatic material comprising: a collagen-like polypeptide comprising a peptide unit represented by Pro-Y-Gly; and thrombin; which hemostatic material is obtained by freeze-drying and is in the form of a sponge, has been reported (JP 2005-74079 A).

### SUMMARY OF THE INVENTION

However, the hemostatic material disclosed in JP 2005-74079 A comprises a blood coagulation factor, thrombin, as an essential component, and the hemostatic function of the collagen-like polypeptide is not known. Further, of course, there is no known hemostatic material using the collagen-like polypeptide.

Further, in cases where a collagen-like polypeptide is used as a hemostatic material, its ease of handling and productivity need to be improved. More specifically, in order to utilize a polypeptide as a functional material, the polypeptide needs to have a property that allows easy processing of the polypeptide into various forms such as a fiber, and to have strength suitable for production and use of the polypeptide. However, because of insufficient molecular weights, collagen-like polypeptides conventionally produced have problems such as difficulty in spinning and insufficient mechanical strength. This is because collagen-like polypeptides, unlike naturally occurring collagen molecules, are composed of simple repeats each comprising only Pro-Y-Gly, and interactions between molecular chains are therefore poor even though they are high-molecular-weight peptides.

In view of the above-described situation, the present invention aims to provide a medical material having high hemostatic ability, which is easy to handle and shows improved productivity.

As a result of intensive study to solve the above problems, the present inventors discovered that a collagen-like polypeptide itself has high hemostatic ability, and succeeded in production of a collagen-like polypeptide having a molecular weight higher than that of a conventional collagen-like polypeptide. By using the high-molecular-weight collagen-like polypeptide as an effective component of a hemostatic material, the present invention was completed.

That is, the present invention is as follows.

[1] A hemostatic material comprising a polypeptide having a peptide fragment represented by General Formula (1) below:

-(Pro-Y-Gly)ₙ- (1)

[wherein in General Formula (1), Y represents hydroxyproline or proline, and n represents an integer of 74 to 171].
[2] The hemostatic material according to item [1], wherein the polypeptide is contained in a form selected from the group consisting of a nanofiber; a woven fabric or non-woven fabric comprising the nanofiber; and a sponge.
[3] The hemostatic material according to item [2], wherein the content of the polypeptide in the nanofiber, woven fabric, non-woven fabric or sponge is 2.5 to 100% by weight.

By the present invention, a high-performance medical material that enables hemostasis in a short time is provided. Further, by using as a material a collagen-like polypeptide which can be easily processed and has excellent versatility, ease of handling and productivity of a hemostatic material can be improved.

### BRIEF DESCRIPTION OF THE DRAWINGS

FIG. 1 is a graph showing comparison of the amount of bleeding from a perforation of a mouse liver in a hemostasis test.

### DESCRIPTION OF EMBODIMENTS

In the present description, when the term “single molecular chain” is used for a collagen-like polypeptide, the term means a state where a polypeptide comprising a peptide fragment composed of repeats of Pro-Y-Gly (wherein Y represents proline or hydroxyproline) is present as a single chain without forming a structure due to interactions between molecular chains, such as a triple helix. Further, when the term “complex” is used for a collagen-like polypeptide, the term means a state where a collagen-like polypeptide (single molecular) chain has a triple-helix structure. In many cases, the collagen-like polypeptide complex further forms a higher-order structure wherein the triple-helix has a branched structure or molecules of the triple helix associate together. Whether the polypeptide has a triple-helix structure or not can be confirmed by measuring the circular dichroism spectrum as described later.

In the present invention, amino acid residues are abbreviated as follows.
Ala: L-alanine residue
Arg: L-arginine residue
Asn: L-asparagine residue
Asp: L-aspartic acid residue
Cys: L-cysteine residue
Gln: L-glutamine residue
Glu: L-glutamic acid residue
Gly: glycine residue
His: L-histidine residue
Hyp: L-hydroxyproline residue
Ile: L-isoleucine residue
Leu: L-leucine residue
Lys: L-lysine residue
Met: L-methionine residue
Phe: L-phenylalanine residue
Pro: L-proline residue
Sar: sarcosine residue
Ser: L-serine residue
Thr: L-threonine residue
Trp: L-tryptophan residue
Tyr: L-tyrosine residue
Val: L-valine residue

In the present description, the amino acid sequence of a peptide chain is described such that the amino acid residue at the N-terminus is positioned in the left side, and the amino acid residue at the C-terminus is positioned in the right side, according to the conventional manner.

### <1> Collagen-like Polypeptide Contained in Hemostatic Material of Present Invention

The hemostatic material of the present invention comprises a collagen-like polypeptide having a peptide fragment represented by General Formula (1) below.

-(Pro-Y-Gly)ₙ- (1)

In General Formula (1), Y represents hydroxyproline or proline, and the hydroxyproline is, for example, 4Hyp, preferably trans-4-hydroxy-L-proline.

Further, in General Formula (1), the repeat number n represents an integer of 74 to 171. That is, compared to conventional collagen-like polypeptides (for example, those obtained by the synthesis method described in JP 2003-321500 A), the collagen-like polypeptide of the present invention has a larger repeat number.

Further, the weight average molecular weight of the collagen-like polypeptide of the present invention is preferably not less than 20,000, more preferably 26,700 to 45,600 per single molecular chain. That is, the collagen-like polypeptide of the present invention has a higher molecular weight than conventional collagen-like polypeptides (for example, collagen-like polypeptides obtained by the synthesis method described in JP 2003-321500 A have weight average molecular weights of about 16000 per single molecular chain).

In the present invention, by using a collagen-like polypeptide having a higher molecular weight per single molecular chain than conventional collagen-like polypeptides, it is possible to provide a hemostatic material comprising a material which has excellent processability that allows processing of the material alone into a fiber or nanofiber and also has improved strength.

In the present description, the weight average molecular weight of a polypeptide is represented as a value per single molecular chain as measured by HFIP GPC under the following conditions. HFIP GPC is a method that allows measurement of the accurate molecular weight of a polypeptide single molecular chain rather than an apparent molecular weight of a triple helix or associated molecules.
Mobile phase: hexafluoroisopropanol
Column: GPC KF-606M, manufactured by Showa Denko K.K.
Flow rate: 0.2 to 1 mL/min.
Temperature: 18 to 50° C.
Molecular weight standards: PHG oligomers, and collagen-like polypeptides whose absolute molecular weights were measured by MALS (Table 1)
Detection: UV spectrophotometer

TABLE 1

| | Theoretical molecular weight | Elution time (minutes) |
|---|---|---|
| Poly-PHG No. 1 | 27,000*¹ | 4.736 |
| Poly-PHG No. 2 | 11,000*¹ | 4.916 |
| (PHG)₁₀ | 3,000*² | 5.213 |
| (PHG)₄ | 1,086*² | 5.418 |
| (PHG)₂ | 522*² | 5.652 |
| PHG | 285*² | 6.148 |

*¹The absolute molecular weight of the single molecular chain was determined by MALS (DAWN HELEOS, manufactured by Wyatt Technology).
*²The theoretical molecular weight was determined by calculation.

Similarly to naturally occurring collagen, the collagen-like polypeptide of the present invention can form a triple-helix structure, to form a collagen-like polypeptide complex. Whether a polypeptide has a triple-helix structure or not can be confirmed by subjecting a polypeptide solution to measurement of the circular dichroism spectrum. More specifically, in cases where a positive Cotton effect is found at a wavelength of 220 to 230 nm, and a negative Cotton effect is found at a wavelength of 195 to 205 nm, the polypeptide is considered to have a triple-helix structure. In cases where the polypeptide is a complex having a triple-helix structure, the polypeptide is in the state of a collagen-like fiber, so that processing such as spinning can be easily carried out.

The collagen-like polypeptide complex of the present invention may be linear, or may have one or more branches. In cases where the polypeptide has a branch, a triple-helix structure may be formed after the branching point, or a branching point may be located after the triple-helix structure. Further, the polypeptide chains may be cross-linked to each other.

The collagen-like polypeptide of the present invention may be composed of only a peptide fragment represented by the General Formula (1) described above, or may additionally comprise an amino acid residue(s), peptide fragment(s), and/or alkylene.

For example, the amino acid residue(s) may be at least one selected from Ala, Arg, Asn, Asp, Cys, Gln, Glu, Gly, His, Hyp, Ile, Leu, Lys, Met, Phe, Pro, Sar, Ser, Thr, Trp, Tyr and Val. Examples of the peptide fragment include peptides wherein a plurality of one or more kinds of the above amino acid residues are linked together. The alkylene may be either linear or branched. Specific examples of the alkylene include, but are not limited to, alkylenes having 1 to 18 carbon atoms, and alkylenes having 2 to 12 carbon atoms are practically preferred.

The single molecular chain of the collagen-like polypeptide of the present invention comprises the peptide fragment represented by the General Formula (1) and the other amino acid residue(s), peptide fragment(s) and/or alkylene at a weight ratio within the range of 1:99 to 100:0, preferably 10:90 to 100:0 (=the peptide fragment represented by the General Formula (1): the other amino acid residue(s), peptide fragment(s) and/or alkylene).

### <2> Method for Producing Collagen-like Polypeptide

The method for producing a collagen-like polypeptide of the present invention is described below, but the method is not limited thereto.

The collagen-like polypeptide of the present invention can be produced by condensation reaction of peptide oligomers represented by any of the General Formulae (4) to (6) described below.

H-(Pro-Y-Gly)ₘ-OH (4)

H—(Y-Gly-Pro)ₘ-OH (5)

H-(Gly-Pro-Y)ₘ—OH (6)

In General Formulae (4) to (6), Y represents hydroxyproline or proline, preferably hydroxyproline. The hydroxyproline is, for example, 4Hyp, preferably trans-4-hydroxy-L-proline. m represents an integer of 1 to 10, and is preferably an integer of 1 to 5 in view of ease of handling, efficiency of the condensation reaction, availability of the peptide oligomer and economic efficiency.

As the peptide oligomers represented by General Formulae (4) to (6), either one type or a mixture of a plurality of types of the peptide oligomers may be used. m may be a single integer, or oligomers having various repeat numbers may be mixed.

These peptide oligomers can be obtained by a known solid-phase synthesis method or liquid-phase synthesis method.

A peptide oligomer(s) (about 1- to 10-mer) other than the peptide oligomer(s) represented by General Formulae (4) to (6) may also be used. In such cases, the ratio between the amount of the peptide oligomer(s) represented by General Formulae (4) to (6) to be used and the amount of the another/other peptide oligomer(s) to be used is preferably within the range of 100:0 to 50:50 in terms of the weight ratio, in view of the capacity of the produced collagen-like polypeptide single molecular chains to form a triple-helix structure and hence to become a complex.

The condensation reaction is carried out in an aqueous solvent comprising 0 to 0.2 M phosphate ions. The aqueous solvent herein is solvent comprising water, and the aqueous solvent may be contaminated with organic solvents. The organic solvents mean amides (dimethylformamide, dimethylacetamide, hexamethylphosphoramide and the like), sulfoxides (dimethylsulfoxide and the like), nitrogen-containing cyclic compounds (N-methylpyrrolidone, pyridine and the like), nitriles (acetonitrile and the like), ethers (dioxane, tetrahydrofuran and the like) and alcohols (methyl alcohol, ethyl alcohol, propyl alcohol and the like). The term “may be contaminated” means that the content of the organic solvents is preferably less than 50% by weight, more preferably less than 10% by weight. The organic solvents are still more preferably not contained at all.

The “phosphate ion” contained in the aqueous solvent is a general term for a dihydrogen phosphate ion (H₂PO₄⁻), hydrogen phosphate ion (HPO₄²⁻) and phosphate ion (PO₄³⁻), and the phosphate ion concentration in the aqueous solvent is the total concentration of dihydrogen phosphate ions (H₂PO₄⁻), hydrogen phosphate ions (HPO₄²⁻) and phosphate ions (PO₄³⁻).

The present inventors discovered that high-molecular-weight collagen-like polypeptides can be produced by decreasing the phosphate ion concentration in the aqueous solvent; that low-molecular-weight collagen-like polypeptides can be produced by increasing the phosphate ion concentration in the aqueous solvent; and that the molecular weight of the collagen-like polypeptide to be produced can be controlled by adjusting the phosphate ion concentration. More specifically, for example, in cases where the concentration of the peptide oligomers in the aqueous solvent is 5% by weight, a collagen-like polypeptide having a weight average molecular weight of 45,600 to 26,700 can be obtained when the phosphate ion concentration is 0 to 0.0025 M, and a collagen-like polypeptide having a weight average molecular weight of 20,300 to 16,000 can be obtained when the phosphate ion concentration is not less than 0.005 M and less than 0.01 M. Thus, collagen-like polypeptides having high molecular weights, which have been conventionally difficult to obtain, can be produced. A collagen-like polypeptide having a weight average molecular weight of 13,500 to 7,100 can be obtained when the phosphate ion concentration is 0.012 to 0.06 M.

The phosphate ion concentration can be adjusted by adding a phosphate such as potassium dihydrogenphosphate or disodium hydrogenphosphate to the aqueous solvent. Since these phosphates can be easily and inexpensively obtained and their concentrations can be easily adjusted, the present invention can be easily carried out with them.

In the condensation reaction, the concentration of the peptide oligomers in the aqueous solvent is preferably 0.1 to 50% by weight in view of the reaction efficiency, and is more preferably 4 to 25% by weight in view of handling of the reaction. It is also possible to control the molecular weight of the collagen-like polypeptide to become smaller by decreasing the concentration of the peptide oligomers.

The temperature at which the condensation reaction is carried out is preferably 0 to 60° C. in view of the reaction efficiency, and is more preferably 4 to 20° C.

The reaction time is preferably 1 to 96 hours, more preferably 2 to 48 hours.

The pH of the aqueous solvent wherein the condensation reaction is carried out is not limited, and is normally adjusted to a neutral or nearly neutral pH (pH of about 6 to 8). The pH may be adjusted using an inorganic base (sodium hydroxide, potassium hydroxide, sodium carbonate, sodium hydrogen carbonate or the like), organic base, inorganic acid (hydrochloric acid or the like) or organic acid.

The aqueous solvent may be stirred for increasing the reaction efficiency, but the stirring is not necessarily required.

The condensation reaction is carried out in the presence of a dehydrating agent (dehydration condensation agent, condensation aid). By allowing the reaction to proceed in the presence of a dehydration condensation agent or condensation aid, the condensation reaction smoothly proceeds without laborious treatment wherein deprotection and amino acid binding are repeated, while dimerization and cyclization are suppressed.

The dehydration condensation agent is not limited as long as the dehydration condensation can be efficiently carried out therewith in the above solvent. Examples of the dehydration condensation agent include carbodiimide condensing agents (diisopropylcarbodiimide (DIPC), 1-ethyl-3-(3-dimethylaminopropyl)-carbodiimide (EDC=WSCI), 1-ethyl-3-(3-dimethylaminopropyl)-carbodiimide hydrochloride (WSCI-HCl), dicyclohexylcarbodiimide (DCC) and the like), fluorophosphate condensing agents (O-(7-azabenzotriazol-1-yl)-1,1,3,3-tetramethyluronium hexafluorophosphate, O-benzotriazol-1-yl-N,N,N′,N′-tetramethyluronium hexafluorophosphate, benzotriazol-1-yl-oxy-tris-pyrrolidinophosphonium hexafluorophosphate, benzotriazol-1-yl-tris(dimethylamino)phosphonium hexafluorophosphide salt (BOP) and the like), and diphenylphosphorylazide (DPPA).

Each of these dehydration condensation agents may be used alone, or two or more of these may be used in combination as a mixture. Among the dehydration condensation agents, carbodiimide condensing agents (1-ethyl-3-(3-dimethylaminopropyl)-carbodiimide, 1-ethyl-3-(3-dimethylaminopropyl)-carbodiimide hydrochloride and the like) are preferred.

In cases where a nonaqueous solvent, which comprises no water, is employed, the amount of the dehydration condensation agent(s) to be used is usually within the range of 0.7 to 5 moles, preferably within the range of 0.8 to 2.5 moles, more preferably within the range of 0.9 to 2.3 moles (for example, 1 to 2 moles) with respect to a total amount of the peptide oligomers of 1 mole. In cases where a solvent comprising water (aqueous solvent) is employed, inactivation of the dehydration condensation agent(s) occurs, so that the amount of the dehydration condensation agent(s) to be used is usually within the range of 2 to 500 moles, preferably within the range of 5 to 250 moles, more preferably within the range of 10 to 125 moles with respect to a total amount of the peptide oligomers of 1 mole.

The condensation aid is not limited as long as it promotes the condensation reaction, and examples of the condensation aid include N-hydroxy polyvalent carboxylic acid imides (for example, N-hydroxydicarboxylic acid imides such as N-hydroxysuccinic acid imide (HONSu) and N-hydroxy-5-norbornene-2,3-dicarboxylic acid imide (HONB)); N-hydroxytriazoles (for example, N-hydroxybenzotriazoles such as 1-hydroxybenzotriazole (HOBt)); triazines such as 3-hydroxy-4-oxo-3,4-dihydro-1,2,3-benzotriazine (HOOBt); and 2-hydroxyimino-2-cyanoacetic acid ethyl ester.

Each of these condensation aids may be used alone, or two or more of these may be used in combination. Among the condensation aids, N-hydroxydicarboxylic acid imides (such as HONSu), N-hydroxybenzotriazoles and N-hydroxybenzotriazines (HOBt) are preferred.

Irrespective of which type of solvent is used, the amount of the condensation aid(s) to be used is usually within the range of 0.5 to 5 moles, preferably within the range of 0.7 to 2 moles, more preferably within the range of 0.8 to 1.5 moles with respect to a total amount of the peptide oligomers of 1 mole.

The dehydration condensation agent(s) and the condensation aid(s) are preferably used in an appropriate combination. Examples of the combination of the dehydration condensation agent(s) and the condensation aid(s) include DCC-HONSu (HOBt or HOOBt) and WSCI-HONSu (HOBt or HOOBt).

### <3> Hemostatic Material of Present Invention

As shown in the later-mentioned Examples, the collagen-like polypeptide of the present invention has blood clotting ability, and hemostatic ability against injury of the living body. The collagen-like polypeptide has these characteristics irrespective of whether the polypeptide is the form of a solution (such as an aqueous solution) or in the form or a solid.

Therefore, the collagen-like polypeptide can be used as a hemostatic material in various forms. The form of the hemostatic material comprising the collagen-like polypeptide of the present invention is not limited, and examples of the form include a nanofiber; a woven fabric or non-woven fabric comprising the nanofiber; and a sponge. Among these, the form as a nanofiber is preferred since a nanofiber has a larger specific surface area and hence higher blood clotting ability and hemostatic ability compared to forms such as a film or block of a collagen-like polypeptide, which have smaller specific surface areas for contacting with blood (this is also shown by their higher specific gravities). Further, a woven fabric or non-woven fabric comprising a nanofiber is preferred in view of production of higher hemostatic ability since it additionally provides ease of handling.

In such cases, the content of the synthetic collagen in the nanofiber; woven fabric or non-woven fabric comprising the nanofiber; or sponge; is preferably 2.5 to 100% by weight in view of securing the hemostatic ability.

Since the molecular weight (per single molecular chain) of the collagen-like polypeptide contained in the hemostatic material of the present invention is high, the material has high mechanical strength also in cases where the material is in the form of a sponge or nanofiber. Therefore, the material is less likely to be deformed or broken even during hemostasis and after use in hemostasis, and hence has advantages in, for example, that hemostasis can be carried out by pressing the hemostatic material placed on the affected area, and that the material is easy to handle after use.

In cases where the hemostatic material is a woven fabric or non-woven fabric, the thickness of the woven fabric or non-woven fabric is preferably 0.01 to 0.5 mm, more preferably 0.1 to 0.3 mm in view of ease of handling.

The hemostatic material in the form of a woven fabric or non-woven fabric preferably has a form wherein a supporting base material is further laminated, in view of further improving the hemostatic ability. The form of the supporting base material is not limited, and examples of the form of the supporting base material include a film, non-woven fabric, gel and plate. The material of the supporting base material is also not limited, and, for example, a polymer such as polyurethane is preferred in view of handling. The supporting base material layer may be formed by an arbitrary method. For example, the supporting base material layer may be formed by laminating/attaching a polyurethane film on one side of a non-woven fabric. The thickness of the supporting base material is preferably 0.01 to 0.2 mm, more preferably 0.1 to 0.3 mm in view of ease of handling and hemostatic ability.

The hemostatic material of the present invention in the form of a nanofiber, woven fabric, non-woven fabric, sponge or the like may be provided as it is for use in hemostasis, but the material may also be processed into a form wherein the material is placed on a support in order to improve its ease of handling.

When the hemostatic material of the present invention is used for hemostasis, the hemostatic material is brought into contact with the affected area that requires hemostasis, such as an abraded wound or incised wound. In such a case, the hemostatic material may be brought into contact with the affected area while the hemostatic material is either pressed or not pressed onto the affected area. Due to the contact of the hemostatic material with blood, the synthetic collagen as a blood coagulation factor is quickly released, and hemostasis can be achieved in a short time of about several seconds to several ten seconds.

The hemostatic material of the present invention has sufficient hemostatic ability even without addition of other blood coagulation factors such as thrombin, but other coagulant drugs, additives and the like may be further added to the hemostatic material as appropriate, and their addition is not limited. For example, hemostatic components such as thrombin, fibrinogen and oxidized cellulose; cell-adhesive proteins such as fibronectin, vitronectin and laminin; aprotinin, aminocaproic acid and tranexamic acid, which have the antifibrinolytic action; and the like; may be added to the hemostatic material of the present invention. Further, various additives such as stabilizers (for example, albumin and amino acids including L-arginine hydrochloride), antimicrobial agents, preservatives, vitamins and physiologically acceptable salts may be added to the hemostatic material of the present invention. Further, a gel base material such as hyaluronic acid may be added to the hemostatic material of the present invention.

A method for obtaining a nanofiber of a collagen-like polypeptide which may be used as the hemostatic material of the present invention by electrospinning is described below, but the method for obtaining the nanofiber is not limited thereto. Electrospinning is preferred since this method can produce a uniform collagen-like polypeptide fiber having a fiber diameter of 5 nm to 50 μm, and can also produce a nanofiber having a nano-level fiber diameter (1 to 1,000 nm), with which a hemostatic material having a large specific surface area can be obtained.

First, a collagen-like polypeptide is dissolved in a solvent to prepare a spinning solution. The solvent is not limited as long as the polypeptide can be dissolved therein and the solvent can be evaporated in the step of spinning, to allow formation of a fiber. Examples of the solvent include water, ethanol, methanol, isopropanol, acetone, sulfolane acetone, propanol, dichloromethane, formic acid, hexafluoroisopropanol, hexafluoroacetone, methyl ethyl ketone, chloroform, isopropanol, toluene, tetrahydrofuran, benzene, benzyl alcohol, 1,4-dioxane, carbon tetrachloride, cyclohexane, cyclohexanone, methylene chloride, phenol, pyridine, trichloroethane, acetic acid, N,N-dimethylformamide, dimethyl sulfoxide, N,N-dimethylacetamide, 1-methyl-2-pyrrolidone, ethylene carbonate, propylene carbonate, dimethyl carbonate, acetonitrile, N-methylmorpholine-N-oxide, butylene carbonate, 1,4-butyrolactone, diethyl carbonate, diethyl ether, 1,2-dimethoxyethane, 1,3-dimethyl-2-imidazolidinone, 1,3-dioxolane, ethyl methyl carbonate, methyl formate, 3-methyloxazolidine-2-one, methyl propionate and 2-methyltetrahydrofuran. Each of these solvents may be used alone, or a plurality of the solvents may be used as a mixture.

The polypeptide concentration in the spinning solution is preferably 0.1 to 10.0% by weight, more preferably 1.0 to 8.0% by weight, still more preferably 3.0 to 6.0% by weight in view of easy formation of continuous fibers.

The collagen-like polypeptide of the present invention can be subjected alone to spinning since it has a high molecular weight, but another polymer may also be used together for preparation of a spinning solution. In such a case, the mechanical strength of the obtained fiber can be increased; the length of the fiber can be increased; and/or various functions can be given to the fiber. Examples of the other polymer include, but are not limited to, polyethylene glycol, polyvinyl alcohol, polypropylene and polystyrene.

The spinning solution may contain an arbitrary component as long as the component does not inhibit spinning. Examples of such a component include adhesives and electrolytes.

Since addition of an adhesion makes the produced fibers contact with each other at contacting points, the nanofibers can be formed into a strong and flexible non-woven fabric that produces less fuzz due to friction. The adhesive is not limited as long as adhesion of the produced nanofibers to each other can be achieved therewith and the adhesive is soluble in the solvent for the spinning solution. Examples of the adhesive include adhesives comprising a hot-melt resin; elastomer adhesives; acrylic adhesives; epoxy adhesives; and vinyl adhesives. Examples of the elastomer adhesives include polychloroprene rubbers, styrene/butadiene rubbers, butyl rubbers, acrylonitrile/butadiene rubbers, ethylene/propylene rubbers, chlorosulfonated polyethylene rubbers and epichlorohydrin rubbers. In cases where an adhesive is added, it is preferably added in an amount of 0.5 to 10% by weight with respect to the polypeptide in the spinning solution.

By adding an electrolyte, the charge density on the surface of the spinning solution can be increased, and, as a result, the spinnability can be improved. The electrolyte is not limited as long as it is soluble in the spinning solution and electrolytically dissociates in the spinning solution. Examples of the electrolyte include sodium chloride, potassium chloride, magnesium chloride, sodium carbonate, sodium hydrogen carbonate, sodium dihydrogen carbonate and magnesium carbonate. In cases where an electrolyte is added, the amount of the electrolyte to be added is preferably at a level where salting-out of the polypeptide in the spinning solution does not occur, and is preferably 0.5 to 10% by weight with respect to the polypeptide in the spinning solution.

Subsequently, the prepared spinning solution is subjected to spinning by means of the well-known electrospinning method. More specifically, while voltage is applied between a nozzle filled with a spinning solution and a collector (substrate), the spinning solution is discharged from the nozzle, and a fiber is collected on the collector. The conditions for electrospinning are not limited, and may be controlled depending on the type of the spinning solution, use of the obtained fiber, and the like. For example, in common conditions for the method of the present invention, the applied voltage may be 5 to 50 kV; the discharge rate may be 0.01 to 5.00 mL/hour; the vertical distance between the nozzle and the collector may be 50 to 300 mm; and the nozzle to be used may have a diameter of 18 to 30 G. In the spinning environment, the relative humidity is preferably 10 to 70%, and the temperature is preferably 10 to 30° C. However, the relative humidity and the temperature do not necessarily need to be controlled.

A sponge of the collagen-like polypeptide which may be used herein as the hemostatic material of the present invention can be obtained by subjecting an aqueous polypeptide solution to freeze-drying under commonly used conditions, but the method for preparing the sponge is not limited.

Although a sponge has a smaller specific surface area than a nanofiber and a woven/non-woven fabric comprising the nanofiber, a sponge is more preferred in some cases in view of the fact that a sponge can be more easily molded.

### EXAMPLES

The present invention is described below in more detail by way of Examples, but the present invention is not limited to these.

### <Preparation of Collagen-like Polypeptide>

### 1. Preparation of High-Molecular-Weight Collagen-Like Polypeptide (HMW-SC)

To 5 mL of pure water, 0.5 g of L-propyl-L-(4-hydroxypropyl)-glycine as a monomer and 0.05 g of HOBt-H₂O were added, and the resulting mixture was stirred at 4° C. In another container, 1.58 g of EDC-HCl was weighed, and the EDC-HCl was added to 5 mL of pure water, followed by stirring the resulting mixture at 4° C. These mixtures were mixed together to initiate the condensation reaction, and the reaction was allowed to proceed at 4° C. for 24 hours.

As a result of measurement of the weight average molecular weight per single molecular chain of the obtained product by HFIP GPC, the weight average molecular weight was 26,700. The measurement conditions for HFIP GPC were: 5 mM CF₃COONa HFIP solution; column, GPC KF-606M; flow rate, 0.5 mL/min; temperature, 40° C. The above-mentioned PHG, (PHG)₂, (PHG)₄ and (PHG)₁₀, and collagen-like polypeptide single molecular chains whose absolute molecular weights were determined by MALS were used as molecular weight standards.

### 2. Preparation of Conventional Collagen-like Polypeptide (SC)

By the synthesis method described in JP 2003-321500 A, a collagen-like polypeptide was prepared. That is, to 5 mL of a PBS solution (aqueous solution of 8.1 mM Na₂HPO₄, 2.68 mM KCl and 1.47 mM KH₂PO₄), 0.5 g of L-propyl-L-(4-hydroxypropyl)-glycine as a monomer and 0.05 g of HOBt-H₂O were added, and the resulting mixture was stirred at 4° C. In another container, 1.58 g of EDC-HCl was weighed, and the EDC-HCl was similarly added to 5 mL of a dilution prepared by diluting the PBS solution, followed by stirring the resulting mixture at 4° C. These mixtures were mixed together to initiate the condensation reaction, and the reaction was allowed to proceed at 4° C. for 24 hours.

As a result of measurement of the weight average molecular weight per single molecular chain of the obtained product by HFIP GPC under the same conditions as described above, the weight average molecular weight was 16,000.

### <Hemostasis Test>

Each of 0.5 w/w % aqueous solution of the thus prepared collagen-like polypeptide HMW-SC (weight average molecular weight per single molecular chain, 26,700); 0.5 w/w % aqueous solution of SC (weight average molecular weight per single molecular chain, 16,000); and an aqueous solution (SC+HA) prepared by mixing 1.6 w/w % aqueous solution of SC with 0.6 w/w % aqueous solution of hyaluronic acid at a ratio of 1:1 (volume ratio); was subjected to freeze-drying at −80° C., and then heated at 180° C. for 2 hours, to obtain a sponge-like thermally cross-linked product. The SC sponge had a specific gravity of 0.007 to 0.064 g/cm³. A hemostasis test was carried out using 6 mg of the thermally cross-linked product of the freeze-dried collagen-like polypeptide sponge. Further, 6 mg of TERUPLUG (registered trademark, manufactured by Olympus Terumo Biomaterials Corp.), which is a hemostatic material composed of thermally cross-linked bovine-derived collagen, was similarly subjected to the hemostasis test for comparison. Further, 6 mg of gauze was similarly subjected to the hemostasis test.

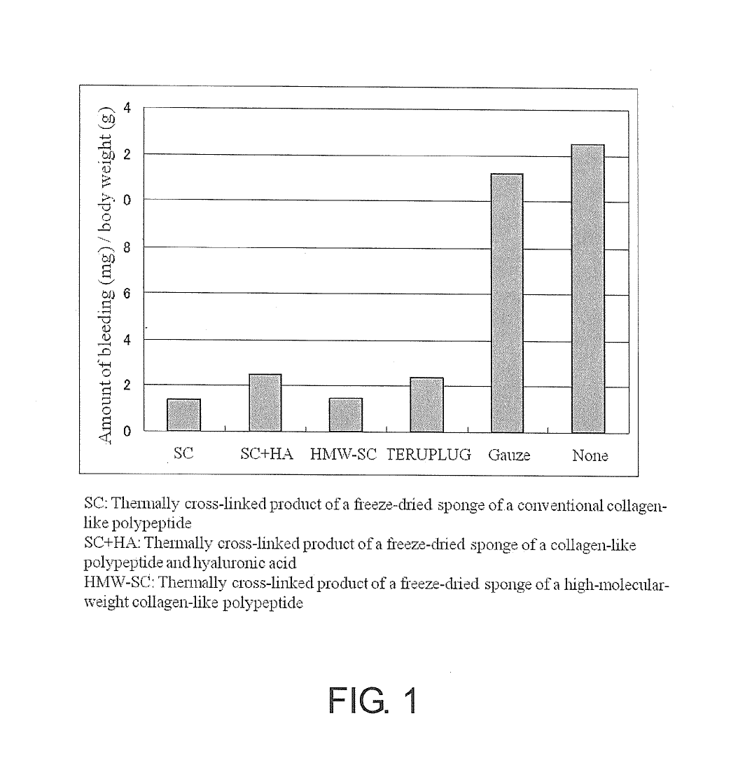

Using a surgical knife, a mouse liver was stabbed to cause bleeding, and it was confirmed that bleeding continues if no treatment is carried out. The sample was brought into contact with the bleeding area such that the entire bleeding area (perforation) was covered, and the liver was left to stand until hemostasis was achieved. No pressure was applied from the upside of the sample. The weight of the sample was measured before and after the hemostasis, and the amount of bleeding was compared. In cases where a hemostatic material was not used, the bleeding area was not covered, and filter paper was used for absorption of blood from the bleeding area. The weight of the filter paper was measured before and after the treatment. As can be seen from the results shown in FIG. 1, it was found that the hemostatic materials comprising a collagen-like polypeptide including the hemostatic material of the present invention enable hemostasis with smaller amounts of bleeding compared to the hemostatic material containing a naturally occurring collagen or the gauze.

Further, the condition of the sponge was observed after the hemostasis test. As can be seen from the results shown in Table 2, in the hemostatic material of the present invention, the strength of the sponge was high, and its shape was maintained even after the absorption of blood.

TABLE 2

| Sample | Condition |
|---|---|
| Collagen-like polypeptide (SC) | The sponge became fragile and was |
| sponge | broken after blood absorption. |
| High-molecular-weight collagen- | The shape did not change even after |
| like polypeptide (HMW-SC) | blood absorption. |
| sponge | |
| TERUPLUG (registered | The shape did not change even after |
| trademark) | blood absorption. |

### <Reference: Test for Confirmation of Blood Coagulation>

In a 24-well plate (manufactured by Sumitomo Bakelite Co., Ltd.; Sumilon multiplate), 500-μL aliquots of pig blood (sodium citrate-treated; purchased from Tokyo Shibaura Zoki Co., Ltd.) were placed, and 3 to 5 μL of a CaCl₂ solution (250 mM) was added to each well to a final Ca²⁺ concentration of 1.5 to 2.5 mM. The plate was shaken (197 rpm) in a shaker (manufactured by TAITEC, BR-40LF) with incubation at 37° C. for 60 seconds, and then incubated in a water bath at 37° C. to measure the time required for coagulation of the blood. The results are shown in Table 3. Based on the results, the final Ca²⁺ concentration was set to 1.5 mM in the later blood coagulation tests.

TABLE 3

| | | | |
|---|---|---|---|
| Amount of CaCl₂ | 3 (n = 2) | 4 (n = 2) | 5 (n = 2) |
| added (μL) | | | |
| Final Ca²⁺ | 1.5 | 2.0 | 2.5 |
| concentration (mM) | | | |
| Coagulation time | 5.5 to 6.5 | 3.5 to 4.0 | 2.0 |
| (minutes) | | | |

### <Reference: Blood Coagulation Test 1>

To each of four wells of a 24-well plate (manufactured by Sumitomo Bakelite Co., Ltd.; Sumilon multiplate), 500 μL of pig blood (sodium citrate-treated; purchased from Tokyo Shibaura Zoki Co., Ltd.) was aliquoted, and 3 μL of a CaCl₂ solution (250 mM) was added thereto to a final Ca²⁺ concentration of 1.5 mM. The plate was shaken (197 rpm) in a shaker (manufactured by TAITEC, BR-40LF) with incubation at 37° C. for 60 seconds, and 165 μL of an aqueous collagen-like polypeptide (SC) solution (0.5 w/w %) was then added to two wells. The plate was left to stand in a water bath incubated at 37° C. to measure the blood coagulation time. The results are shown in Table 4. Based on the results, it was confirmed that addition of the collagen-like polypeptide solution slightly promotes blood coagulation.

TABLE 4

| | | |
|---|---|---|
| Addition of collagen-like | No (n = 2) | Yes (n = 2) |
| polypeptide (SC) | | |
| Time (minutes) | 4.5 to 5.5 | 3.5 to 4.0 |

### <Blood Coagulation Test 2: Solution>

To each of two wells of a 24-well plate (manufactured by Sumitomo Bakelite Co., Ltd.; Sumilon multiplate), 500 μL of pig blood (sodium citrate-treated; purchased from Tokyo Shibaura Zoki Co., Ltd.) was aliquoted, and 3 μL of a CaCl₂ solution (250 mM) was added thereto to a final Ca²⁺ concentration of 1.5 mM. The plate was shaken (100 rpm) in a shaker (manufactured by TAITEC, BR-40LF) with incubation at 37° C. for 30 seconds, and 165 μL of an aqueous SC or HMW-SC collagen-like polypeptide solution (0.5 w/w %) was added to each well. The plate was shaken (197 rpm) at 37° C. for 20 seconds, and then gently shaken (50 rpm) at 37° C. while the condition of the blood was observed every minute. The results are shown in Table 5. Based on the results, it was confirmed that addition of each of the collagen-like polypeptides promotes blood coagulation.

TABLE 5

| Time | Collagen-like polypeptide | | |
|---|---|---|---|
| (minutes) | None (n = 1) | SC (n = 1) | HMW-SC (n = 1) |
| 1 to 3 | — | — | — |
| 4 | — | Δ | Δ |
| 5 | — | ◯ | ◯ |
| 6 to 9 | — | ◯ | ◯ |
| 10 | Δ | ◯ | ◯ |

—: No change,
Δ: Initiation of coagulation,
◯: Coagulation

### <Blood Coagulation Test 3: Sponge>

Each of the SC and HMW-SC collagen-like polypeptides and a naturally occurring collagen (Nippon Meat Packers, Inc.; pig skin; 1%; pH 3; aqueous hydrochloric acid solution) was prepared into a 0.5 w/w % aqueous solution, and freeze-dried to obtain a sponge-shaped sample. Test tubes each containing 0.1 g of the sample, and an empty test tube for a control were provided, and 1 mL of pig blood (2 days after collection; sodium citrate-treated; purchased from Tokyo Shibaura Zoki Co., Ltd.) was added to each test tube, immediately followed by addition of 3 μL of a 250 mM calcium chloride solution thereto. Observation was carried out every 15 to 30 seconds by tilting each test tube to measure the time required for blood coagulation. The results are shown in Table 6.

TABLE 6

| | Coagulation time |
|---|---|
| Sample | (minutes) |
| Collagen-like polypeptide (SC) sponge | 5.3 |
| Collagen-like polypeptide (HMW-SC) | 5.1 |
| sponge | |
| Naturally occurring collagen sponge | 8.0 |
| Control (no sample) | 8.5 |

### <Reference: Blood Coagulation Test 4: Film>

In an ice bath, 4 g of an SC collagen-like polypeptide solution (0.615 w/w %) was cooled, and 75 μL of 0.01N NaCl, 0.26 mM NaHCO₃ and 20 mM HEPES cooled in an ice bath were added to the solution. The resulting solution was placed in a Teflon (registered trademark) round container (diameter, 5 cm), and subjected to drying in an incubator at 35° C., to obtain a circular SC film (62.1 mg). The film had a specific gravity of 3.2 g/cm³.

To 5 g of a naturally occurring collagen (NC) solution (Nippon Meat Packers, Inc.; pig skin; 1%; pH 3; aqueous hydrochloric acid solution), 5 mL of ultrapure water was added, and the resulting dilution was cooled in an ice bath. To the cooled dilution, 150 μL of 0.01N NaCl, 0.26 mM NaHCO₃ and 20 mM HEPES cooled in an ice bath were added, to adjust the pH to 6. The resulting solution was aliquoted in 5-mL volumes into two Teflon (registered trademark) round containers (diameter, 5 cm), and dried in an incubator at 35° C., to obtain circular NC films (67.2 mg).

In a 24-well plate (manufactured by Sumitomo Bakelite Co., Ltd.; Sumilon multiplate), 500-μL aliquots of pig blood (sodium citrate-treated; purchased from Tokyo Shibaura Zoki Co., Ltd.) were placed, and 2 μL of a CaCl₂ solution (250 mM) was added to the blood in each well to a final Ca²⁺ concentration of 1.5 mM. The plate was shaken (100 rpm) in a shaker (manufactured by TAITEC, BR-40LF) with incubation at 37° C. for 30 seconds. The SC film (1.1 mg) or NC film (1.1 mg) was added to each well, and the plate was then vigorously shaken (197 rpm) at 37° C. for 20 minutes. Thereafter, the plate was gently shaken at 50 rpm while the condition of the blood was observed every minute. The results are shown in Table 7 and Table 8. Although the coagulation time was different between the first and second tests, whether a film was used or not did not cause any difference in the blood coagulation time. Blood coagulation took longer in the cases where a film was used compared to the cases where a sponge was used.

TABLE 7

| Time | | | |
|---|---|---|---|
| (minutes) | No film | SC film | NC film |
| 1 to 8 | — | — | — |
| 9 | Δ | Δ | Δ |
| 12 | ◯ | ◯ | ◯ |

—: No change,
Δ: Initiation of coagulation,
◯: Coagulation,
↓: Progression of coagulation

TABLE 8

| Time | | | |
|---|---|---|---|
| (minutes) | No film | SC film | NC film |
| 1 to 6 | — | — | — |
| 7 | Δ | Δ | — |
| 8 | ↓ | ↓ | Δ |
| 9 | ◯ | ◯ | ◯ |

—: No change,
Δ: Initiation of coagulation,
◯: Coagulation,
↓: Progression of coagulation

### INDUSTRIAL APPLICABILITY

By the present invention, a high-performance medical material that enables hemostasis in a short time is provided. Further, since use of a collagen-like polypeptide which can be easily processed and has excellent versatility as a material improves ease of handling and productivity of a hemostatic material, the material is industrially very useful.

While the invention has been described in detail with reference to preferred embodiments thereof, it will be apparent to one skilled in the art that various changes can be made, and equivalents employed, without departing from the scope of the invention. Each of the aforementioned documents is incorporated by reference herein in its entirety.

1. A hemostatic material comprising a polypeptide having a peptide fragment represented by General Formula (1) below:
-(Pro-Y-Gly)ₙ- (1)
[wherein in General Formula (1), Y represents hydroxyproline or proline, and n represents an integer of 74 to 171].

2. The hemostatic material according to claim 1, wherein said polypeptide is contained in a form selected from the group consisting of a nanofiber; a woven fabric or non-woven fabric comprising said nanofiber; and a sponge.

3. The hemostatic material according to claim 2, wherein the content of said polypeptide in said nanofiber, woven fabric, non-woven fabric or sponge is 2.5 to 100% by weight.
